SEQUENCE LISTING (6 sequences)

SEQ ID NO: 1 - DNA, 2075 bp, homo sapiens
cccggccgacagccccgagacgacagcccggcgcgtcccggtccccacctccgaccaccgccagcgctccaggccccgcgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcttccccagacactggtttgaagaatgttaagacttgacagtggaactacattagtacacagcaccagaatgtatattaaggtgtggctttaggagcagtgggagggtaccggcccggttagtatcatcagatcgactcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtcttggcaggctgatttctaggtaggaaatgtggtagctcacg

SEQ ID NO: 2 - protein, 349 aa, homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 3 - DNA, 23 bp, homo sapiens
cagctctgacattctgattcgaa

SEQ ID NO: 4 - DNA, 20 bp, homo sapiens
tgccacaagctgtccagtct

SEQ ID NO: 5 - DNA, 21 bp, homo sapiens
gtccctgccctttgtacacac

SEQ ID NO: 6 - DNA, 19 bp, homo sapiens
cgatccgagggcctcacta

(19) United States
(12) Patent Application Publication
(10) Pub. No.: US 2006/0275797 A1
(43) Pub. Date: Dec. 7, 2006

(54) Use of agents which inhibit connective tissue growth factor (CTGF) binding and signaling via the TrkA/p75NTR receptor complex for the prevention and treatment of CTGF-mediated ocular disorders

(75) Inventors: Allan R. Shepard, Fort Worth, TX (US); Nasreen Jacobson, Fort Worth, TX (US); Abbot F. Clark, Arlington, TX (US)

Correspondence Address: ALCON, IP LEGAL, TB4-8, 6201 SOUTH FREEWAY, FORT WORTH, TX, 76134, US

(73) Assignee: Alcon Manufacturing, Ltd.

(21) Appl. No.: 11/384,638

(22) Filed: Mar. 20, 2006

Related U.S. Application Data
(60) Provisional application No. 60/663,854, filed on Mar. 21, 2005.

(51) Int. Cl. C12Q 1/68; A61K 38/17

(52) U.S. Cl. 435/6; 514/12

(57) ABSTRACT

The present invention provides a method for lowering intraocular pressure and providing neuroprotection to a patient in need thereof by administering a therapeutically effective amount of at least one agent that inhibits binding and/or signaling of connective tissue growth factor (CTGF) via the TrkA/p75^{NTR} receptor complex.

The present application claims the benefit of U.S. Provisional Patent Application having Ser. No. 60/663,854 filed Mar. 21, 2005.

### BACKGROUND OF THE INVENTION

1. Field of the Invention

The present invention relates to the field of ocular conditions mediated by the elevation of connective tissue growth factor (CTGF) in ocular tissues. More specifically, the invention provides compositions that prevent and/or treat such CTGF-mediated ocular disorders.

2. Description of the Related Art

There are a number of ocular conditions that are caused by, or aggravated by, damage to the optic nerve head, degeneration of ocular tissues, and/or elevated intraocular pressure. For example, “glaucomas” are a group of debilitating eye diseases that are a leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3%. of all adults over 40 years old (Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Twenty years ago, the interplay of ocular hypertension, ischemia and mechanical distortion of the optic nerve head were heavily debated as the major factors causing progression of visual field loss in glaucoma. Since then, other factors including excitotoxicity, nitric oxide, absence of vital neurotrophic factors, abnormal glial/neuronal interplay and genomics have been implicated in the degenerative disease process. Neurotrophins and their receptors have been implicated in the normal homeostasis of the RGC and Lamina Cribrosa of the eye. Neurotrophins consist of nerve growth factor (NGF), brain-derived neurotrophic factor (BDNF), and neurotrophin 4 (NT-4) and bind to the high affinity TrkA, TrkB, and TrkC receptors, respectively. NGF and NT-4 are also known to bind to the low affinity p75^{NTR} receptor. Most studies on RGC's and glaucoma, to date, have examined the role of TrkB receptors and its ligand BDNF. BDNF is known to be important for RGC cell survival in vitro and in vivo. However, innapropriate neurotrophin expression and stimulation may be one mechanism of cell death in POAG. TrkA expression in RGC's may be upregulated in experimental glaucoma (Rudzinski et al. (2004); Cui et al., (2002)). This has been theorized to provide a neuroprotective role under stressful conditions; however, aberant stimulation of TrkA may, in theory, lead to aberant NTR signaling resulting in apoptosis of RGC's or TM cells.

The consideration of genomics deserves some discussion insofar as it may ultimately define the mechanism of cell death, and provide for discrimination of the various forms of glaucoma. Within the past 8 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congentical glaucoma (CYP1B1), two mapped genes for pigmentary dispersion/pigmentary glaucoma, and a number of genes for developmental or syndromic forms of glaucoma (FOXC1, PITX2, LMX1B, PAX6).

Thus, each form of glaucoma may have a unique pathology and accordingly a different therapeutic approach to the management of the disease may be required. For example, a drug that effects the expression of enzymes that degrade the extracellular matrix of the optic nerve head would not likely prevent RGC death caused by excitotoxicity or neurotrophic factor deficit. In glaucoma, RGC death occurs by a process called apoptosis (programmed cell death). It has been speculated that different types of insults that can cause death may do so by converging on a few common pathways. Targeting downstream at a common pathway is a strategy that may broaden the utility of a drug and increase the probability that it may have utility in the management of different forms of the disease. However, drugs that effect multiple metabolic pathways are more likely to produce undesirable side-effects. With the advent of gene-based diagnostic kits to identify specific forms of glaucoma selective neuroprotective agents can be tested with the aim of reducing the degree of variation about the measured response.

Current glaucoma therapy is directed to lowering IOP, a major risk factor for the development and progression of glaucoma. These therapies lower IOP, but they do not directly address the pathogenic mechanisms, and the disease continues to progress. Thus, what is needed is a therapeutic method for lowering IOP and/or providing neuroprotection to the optic nerve head and/or to retinal ganglion cells via pathogenic pathways.

### SUMMARY OF THE INVENTION

The present invention overcomes these and other drawbacks of the prior art by providing a method for lowering intraocular pressure and providing neuroprotection to a patient in need thereof by administering a therapeutically effective amount of a composition including at least one agent that inhibits binding and/or signaling of connective tissue growth factor (CTGF) mediated via the TrkA/p75^{NTR} receptors, and a pharmaceutically acceptable carrier. Such treatments will be particularly suited for those patients shown to suffer from CTGF-mediated ocular disorders. Determination that a patient suffers from a CTGF-mediated ocular disorder may be made by routine diagnostic assays to detect an abnormally high expression of CTGF gene or its gene product in ocular tissues. CTGF gene or its gene product refers to all genetic elements that are necessary for the transcription, is translation, and overall expression of CTGF including any RNA splicing variants or protein variants or fragments.

Thus, in one aspect, the invention provides a method of treating a patient suffering from a CTGF-mediated ocular disorder by first determining, by routine diagnostic assay, that the patient exhibits abnormally high expression of CTGF in ocular tissues as compared to the amount of CTGF present in normal tissues, and administering to patients exhibiting an abnormally high expression a therapeutically effective amount of a composition containing at least one agent that inhibits CTGF binding or signaling via the TrkA/p75^{NTR} receptor complex.

In another aspect, the invention provides a method for lowering intraocular pressure by administering to a patient a therapeutically effective amount of an agent that inhibits binding and signaling of CTGF mediated via the TrkA/p75^{NTR} receptors. Preferably, the compositions for use in the method of the invention will lower intraocular pressure that is elevated due to an increased expression of CTGF or of a product of CTGF signaling.

In preferred embodiments, the composition of the invention may be administered by topical application, intracamerally or via an implant. Typically, the total concentration of the CTGF inhibitor in the composition of the invention will be from 0.01% to 2%. Generally, the treatment method of the invention will be most useful for a patient suffering from glaucoma, for example normal-tension glaucoma, or ocular hypertension.

The invention further provides a method for preventing the visual field loss associated with POAG by administering to a patient in need thereof a composition including a non-nucleotide or non-protein agent that inhibits binding and/or signaling of CTGF activity mediated via the TrkA/p75^{NTR} receptors, such that intraocular pressure is controlled and protection is provided to retinal ganglion cells or to the optic nerve head.

In another embodiment, the present invention provides a composition for lowering intraocular pressure and providing neuroprotection in a patient in need thereof. Generally, the composition of the invention includes at least one agent that inhibits the binding and/or signaling of CTGF activity mediated via the TrkA/p75^{NTR} receptors and a pharmaceutically acceptable carrier. The total concentration of TrkA/p75^{NTR} inhibitor in the composition of the invention will preferably be from 0.01% to 2%.

The preferred compound for use in the embodiments of the invention described above is the selective alkaloid-like kinase inhibitor, K252a.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. CTGF gene expression is elevated in glaucomatous vs. normal TM tissues. To verify the results of the microarray and cDNA subtraction, CTGF mRNA expression was determined by QPCR analysis of normal vs. glaucoma TM tissue cDNA. The numbers below each sample refer to the cDNA identification number assigned to each sample. Human donor tissue and total RNA were obtained as described (Wang et al. 2001). “Ave.” represents the mean of normal or glaucoma TM levels.

FIG. 2. CTGF gene expression is elevated in glaucomatous vs. normal TM cell lines. To verify the results of the microarray and cDNA subtraction, CTGF mRNA expression was determined by QPCR analysis of normal vs. glaucoma TM cell line cDNA. The numbers below each sample refer to the cell line identification number. Human TM cell lines and total RNA were obtained as described (Shepard et al. 2001). “NTM pool” and “GTM pool” refer to amplification levels from pooled NTM or GTM cDNA.

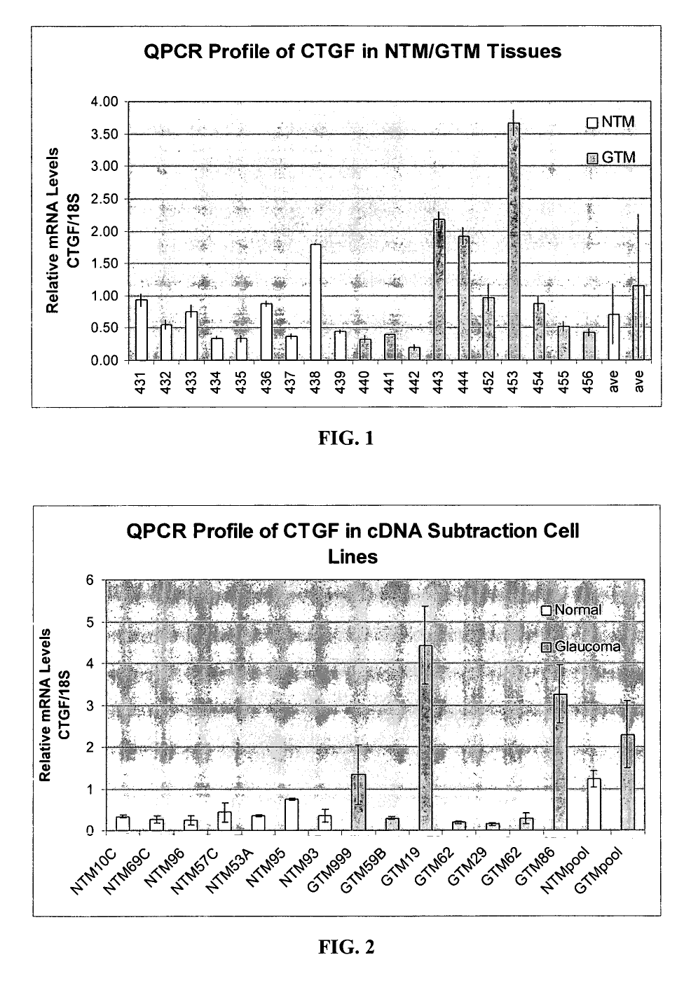

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONE glial cells. In response to elevated IOP and/or transient ischemic insults, there is a change in the composition of the ONH extracellular matrix and alterations in the glial cell and retinal ganglion cell axon morphologies.

Glaucomatous changes to the TM differ from fibrosis, which is associated with a wound healing response and generally involves inflammation and the subsequent proliferation of myofibroblasts. Tissue injury is recognized by the inflammatory system, which initiates a wound repair process by stimulating fibroblasts and angiogenesis. Dead or dying tissues/cells are replaced by scar tissue consisting initially of fibrin, which is subsequently replaced by excessive amounts of extracellular matrix material, particularly collagen.

CTGF is a secreted cytokine which is known to increase extracellular matrix (ECM) production, primarily via increased deposition of collagen I and of fibronectin. Overexpression of CTGF has previously been implicated as a major causative factor in conditions such as scleroderma, fibroproliferative diseases, scarring, etc. in which there is an overaccumulation of ECM components. An overaccumulation of extracellular matrix materials in the region of the trabecular meshwork (TM) is also a hallmark of many forms of glaucoma; such increases are believed to lead to increased resistance to aqueous outflow, and therefore elevated intraocular pressures. It is known that CTGF gene products are present in isolated tissues and cell lines established from human TM. Thus, it is believed that CTGF plays a role in ECM production by the TM. As described in co-owned application Ser. No. 10/510,585, agents which down-regulate CTGF gene expression, protein production, or down-stream effects of CTGF activation, represent a novel means for lowering IOP. That application does not discuss inhibiting the interaction of CTGF with its receptor, TrkA/p75^{NTR}.

CTGF expression can be induced by a wide variety of factors including: TGF-β, VEGF, thrombin, advanced glycation end-products (AGE), mechanical stress, and lysophosphatidic acid (LPA), among others. The specific inducers of CTGF and the signaling pathways can vary depending on the specific cell type being stimulated. There are reports of TGF-β induction of CTGF involving Smads, PLC, PKC, and tyrosine kinases in some cell types while RhoA, PKA, and the cytoskeleton appear to be involved in other cell types. Mechanical stress of fibroblasts induces CTGF expression via involvement of protein kinases and tyrosine phosphatases.

The mechanism of action of CTGF is not well understood. CTGF appears to bind to PDGF receptors, integrins, and LDL receptor-related proteins (LRP), each of which may act as a signaling cell surface receptor for CTGF. In some cell types, CTGF signaling through PDGF receptors appears to involve MAPK and P13K. CTGF signaling in chondrocytes involves ERK signaling for cell proliferation and p38MAPK signaling for cellular differentiation. The specific cell surface receptors and signaling pathways used by CTGF appear to be dependent on the specific cell type being studied.

U.S. Pat. No. 5,585,270 describes polynucleotides encoding CTGF. CTGF is said to have mitogenic activity, or the ability to stimulate target cells to proliferate. It is also said to have chemotactic activity, that is, the chemically induced movement of cells as a result of interaction with particular molecules. The protein is believed to play a role in the normal development, growth and repair of human tissue. The patent also describes a method for accelerating wound healing in a subject by applying to the wound an effective amount of a composition containing purified CTGF. The polypeptide is said to be useful in cases where there is impaired healing of skin wounds or there is a need to augment the normal healing mechanisms, e.g., burns. The patent contains no discussion of glaucoma or eye disorders.

U.S. Pat. No. 6,069,006, whose specification is a continuation-in-part of U.S. Pat. No. 5,585,270 discussed above, describes CTGF regulatory nucleic acid sequences. This patent further describes methods for treating fibrotic diseases and for identifying agents for treatment of fibrotic diseases. No specific agents, other than CTGF nucleic acid sequences or polypeptides are described. Neuroprotection of ocular tissues is not described.

U.S. Pat. No. 6,358,741 describes a number of nucleic acid sequences derived from CTGF that are said to be useful for inhibiting the expression of CTGF in a cell. This patent does not discuss glaucoma, neuroprotection of ocular tissues or inhibition of CTGF binding and/or signaling via the TrkA/p75^{NTR} receptor complex.

CTGF appears to accrue in high concentrations in the cytoplasm of stellate reactive astrocytes of the central nervous system. It has been implicated as a causative agent in mechanisms associated with reactive astrocytosis such as gliosis (glial overgrowth) and glial scar formation, i.e., in processes known to hinder neural repair and growth (Schwab et al. 2000; Schwab et al. 2001). It is believed that such processes also participate in the loss of retinal neurons and/or the inability to repair optic nerve axons associated with glaucoma.

It has now been shown that TrkA/p75^{NTR} is the CTGF receptor in human mesangial cells (Abdel Wahab et al., (2005)). As described above, CTGF expression is induced by TGFβ and acts as a central mediator in ocular disorders such as CNV, AMD, DR, PDR ocular fibrosis and glaucoma. Neurotrophins and their receptors, including TrkA and p75^{NTR} are present in many ocular tissues and have been shown to play an important role in homeostasis. Targeting the downstream effects of CTGF action mediated by TrkA/p75^{NTR} complex has not been disclosed and may provide a more specific therapeutic target and larger therapeutic index than targeting the more general upstream processes.

Rudzinski et al. (2004) disclosed the general use of Trk receptor agonists and p75^{NTR} antagonists as possible therapeutics for ocular hypertension. Rudzinski proposed that the Nerve Growth Factor-TrkA axis may be protective in high IOP situations. However, the present inventors believe that binding of CTGF to the TrkA/p75^{NRT} receptor complex in a pathological situation is deleterious. Therefore, the present invention is directed to the use of antagonists of CTGF binding to the TrkA/p75^{NTR} receptor complex.

Thus, in one aspect, the present invention provides a method for lowering IOP and providing neuroprotection to retinal ganglion cells by administering a composition including is an inhibitor of the CTGF-activated TrkA/p75^{NTR} signaling pathway. In one embodiment, the invention includes first confirming the presence of a CTGF-mediated ocular disorder by obtaining an ocular fluid or tissue sample—such as tear fluid, aqueous humor, vitreous humor or trabecular meshwork, from a patient and determining the amount of CTGF expressed in the tissue sample using routine diagnostic assays. Tear fluid has been shown to contain measurable amounts of CTGF (van Setten et al., (2003)). Another option is to obtain a sample of DNA from the patient, e.g. cheek swab or blood, followed by genetic screening for single nucleotide polymorphisms in any genetic element that may be indicative of elevated CTGF levels in the eye.

The diagnostic assay used may be any assay routine to the skilled artisan that will indicate the amount of CTGF expressed in the sample tissue as compared to the expression in normal control tissues. Preferred assays include ELISA, QPCR, DNA Sequencing, SSCP, SNP microarray, The presence of a CTGF-mediated ocular disorder is confirmed by comparing the amount of CTGF expressed in the sample tissue with the amount of CTGF expressed in normal (i.e., non-glaucomatous) tissues. Once the presence of a CTGF-mediated ocular disorder is confirmed, a composition containing at least one inhibitor of the CTGF-activated TrkA/p75^{NTR} signaling pathway is administered to the patient. Of course, on subsequent administrations to the same patient, the diagnostic assay will no longer be necessary.

The therapeutic agent for use in the present invention may be a peptide, peptidomimetic, small molecule or any other form of therapeutic agent, such as nucleotide sequence, siRNA, etc. Preferably, the therapeutic agent will be a peptide, peptidomimetic or small molecule.

Turner et al. (2004) showed antagonism of p75^{NTR} ligand binding in mouse motor neuron-like cell cultures using a short peptide. US Patent Application No. 20030211982 discloses beta turn peptidomimetic cyclic compounds targeting neurotrophin receptors including TrkA for treating “ocular nerve diseases such as glaucoma.” Neither of the above references discloses the use of agents that inhibit CTGF-activated TrkA/p75^{NTR} signaling in treating CTGF-mediated ocular disorders.

The therapeutic agent for the treatment of glaucoma will preferably be a small drug-like molecule, which affects one or more aspects of the CTGF pathway. The selective alkaloid-like kinase inhibitor, K-252a, is one preferred compound for use in the present invention (Turner et al. (2004); Berg et al (1992).

The agents of this invention, can be incorporated into various types of ophthalmic formulations for delivery to the eye (e.g., topically, intracamerally, or via an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art. The following are examples of possible formulations embodied by this invention.

(a) Topical ocular formulation

| | wt. % |
|---|---|
| TrkA/p75^{NTR} Inhibitor | 0.005-5.0 |
| Tyloxapol | 0.01-0.05 |
| HPMC | 0.5 |
| Benzalkonium chloride | 0.01 |
| Sodium chloride | 0.8 |
| Edetate disodium | 0.01 |
| NaOH/HCl | q.s. pH 7.4 |
| Purified water | q.s. 100 mL |

It is further contemplated that the compounds of the invention could be formulated in intraocular insert devices.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Expression of CTGF in Glaucomatous TM vs Non-Glaucomatous TM

### Microarray Screen

The mRNA transcript for CTGF was identified as being elevated in glaucomatous TM cells vs. normal TM cells by a GeneFilter® (Research Genetics) screen.

Five hundred ηg of total RNA (TRIzol Reagent; Invitrogen) was isolated from pooled (6 each) normal or glaucomatous TM cell lines as described (Shepard et al., IOVS 2001, 42:3173) and reverse-transcribed separately in the presence of 300 units of Superscript II RNase H— reverse transcriptase (Invitrogen), 100 μCi [α-³³P]dCTP (10 mCi/ml, 3,000 Ci/mmol; Amersham), 0.33 mM dATP, dGTP, dCTP (Promega), 3.3 mM DTT, 1× First Strand Buffer (Invitrogen), and 2 μg oligo-dT at 37° C. for 90 min. The radiolabeled cDNA was subsequently purified with Chroma-Spin 100 Columns (Clontech) according to the manufacturer's instructions. A single Genefilter® (GF211; Research Genetics) spotted with ≈4,000 known genes was used for hybridization with radiolabeled probe according to the manufacturer's instructions. The membrane was first pre-treated by boiling in 0.5% SDS for 5 min. Prehybridization of the membrane was performed for 30 min at 40° C. in roller bottles containing 5 ml MicroHyb (Research Genetics) with 5 ug poly-dA (Research Genetics) and 5 μg boiled Cot1 DNA (Invitrogen) as blocking reagents. The entire radiolabeled probe was boiled and added to the prehybridization mixture for 16 h at 42° C. Following hybridization, the membranes were washed twice in 2×SSC/1% SDS for 20 min and then once in 0.5×SSC/1% SDS for 15 min. The membrane was placed on wet Whatmann 3 MM paper and wrapped in SaranWrap. Images were acquired on a Storm Phosphorimager (Molecular Dynamics) using maximum resolution. Once the image was acquired the blot was immediately stripped in boiling 0.5% SDS and subjected to another hybridization with the opposite probe, e.g. normal TM followed by glaucomatous TM. Images from duplicate probings were analyzed using Pathways (Research Genetics) and MS Excel (Microsoft) software. Of the 4300 genes on the GF211 GeneFilter®, CTGF (GenBank accession #AA598794) was one of 7 genes that showed a GTM:NTM ratio >2-fold.

### cDNA Subtraction Screen

CTGF was identified independently in a custom PCR-Select™ cDNA Subtraction screen (Clontech) used to screen for mRNA transcripts that are differentially expressed in glaucomatous vs. normal TM cells.

Seven hundred μg of total RNA (TRIzol Reagent; Invitrogen) was isolated from pooled (7 each) normal or glaucomatous TM cell lines as described (Shepard et al. 2001). Tester (glaucomatous) and driver (normal) poly A+RNA was isolated by two rounds of poly A+ selection on oligo-dT latex beads using a Nucleotrap mRNA Midi kit (Clontech). All subsequent PCR-Select™ cDNA Subtraction steps were performed according to Clontech kit (catalog #K1804-1) methodology.

Differential screening of the resulting cDNA subtraction libraries was performed essentially according to instructions in the PCR-Select™ Differential Screening Kit (Clontech catalog #K1808-1). A select number of differentially expressed cDNA clones were also confirmed by virtual Northern analysis. A list of all the candidate differentially expressed clones identified with subtracted TM cell cDNA library probe was generated by Clontech. The transcript for CTGF (GenBank accession #XM_{—}037055.1) was in this list and was enriched in the glaucomatous cDNA library.

### EXAMPLE 2

### RNA Isolation and First Strand cDNA Preparation

Total RNA was isolated from TM cells using Trizol® reagent according to the manufacturer's instructions (Life Technologies). First strand cDNA was generated from 1 ug of total RNA using random hexamers and TaqMan® Reverse Transcription reagents according to the manufacturer's instructions (PE Biosystems, Foster City, Calif.). The 100 ul reaction was subsequently diluted 20-fold to achieve an effective cDNA concentration of 0.5 ng/ul.

### Quantitative PCR

Differential expression of CTGF was verified by quantitative real-time RT-PCR (QPCR) using an ABI Prism® 7700 Sequence Detection System (Applied Biosystems). Essentially as described (Shepard et al., IOVS 2001, 42:3173). Primers for CTGF amplification were designed using Primer Express software (Applied Biosystems) and anneal to adjacent exons of Genbank accession #NM_{—}001901.1 (CAGCTCTGACATTCTGATTCGAA, nts 1667-1689 and TGCCACAAGCTGTCCAGTCT, nts 1723-1742) and generate a 76-bp amplicon. Amplification of CTGF was normalized to 18S ribosomal RNA expression using primers designed to the 18S rRNA gene (GenBank accession #X03205 GTCCCTGCCCTTTGTACACAC, nts 1680-1700 and CGATCCGAGGGCCTCACTA, nts 1730-1749) which generates a 69-bp amplicon. The SYBR® Green I double-stranded DNA binding dye chemistry (Applied Biosystems) was used to amplify CTGF or 18S cDNA. Specificity of the CTGF and 18S primer pairs was assessed from the PCR product by a combination of DNA sequencing, agarose gel analysis and dissociation curve analysis using the ABI Prism 770 SDS Dissociation Curve software (Applied Biosystems). CTGF or 18S rRNA reactions consisted of 1×SYBR Green PCR Master Mix (Applied Biosystems), 50 nM primer concentrations and 2.5 ng cDNA in a final volume of 50 ul. Thermal cycling conditions consisted of 50° C., 2 min, 95° C. 10 min followed by 40 cycles at 95° C., 15 sec, 60° C., 1 min. Quantification of relative RNA concentrations was done using the relative standard curve method as described in PE Biosystems User Bulletin #2 (http://docs.appliedbiosystems.com/pebiodocs/04303859.pdf). Data analysis was performed with SDS software version 1.9.1 (Applied Biosystems) and MS Excel 97 (Microsoft). Plasmid DNA containing target sequence for CTGF or 18S was used for generating the standard curve. QPCR data are presented as mean±SD.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents and Applications

U.S. Pat. No. 5,585,270

U.S. Pat. No. 6,069,006

U.S. Pat. No. 6,358,741

Ser. No. 10/510,585

Books
Vaughn, D. et al., In: GENERAL OPHTHALMOLOGY, Appleton & Lange, Norwalk, Conn., pp. 213-230 (1992).

Other Publications
Abdel Wahab, N., B. S. Weston, et al., “Connective tissue growth factor CCN2 interacts with and activates the tyrosine kinase receptor TrkA” J AM SOC NEPHROL 161:340-351 (2005).
Bengtsson, B., “Incidence of manifest glaucoma,” BR. J. OPHTHALMOL. 73:483-487 (1989).
Berg et al., “K-252a inhibits nerve growth factor-induced trk proto-oncogene tyrosine phosphorylation and kinase activity,” J BIOL. CHEM. 267:13-16 (1992).
Cui, Q., L. S. Tang, et al., “Expression of trkA, trkB, and trkC in injured and regenerating retinal ganglion cells of adult rats,” INVEST OPHTHALMOL VIS SCI 43:1954-1964 (2002).
Rudzinski, M., T.-P., Wong, et al., “Changes in retinal expression of neurotrophins and neurotrophin receptors induced by ocular hypertension,” J NEUROBIOL 58:341-354 (2004).
Schwab, J. M., et al., “Connective Tissue Growth Factor is Expressed by a Subset of Reactive Astrocytes in Human Cerebral Infarction,” NEUROPATHOL. APPL. NEUROBIOL. 26(5):434-440 (2000).
Schwab, J. M., et al., “Differential Cellular Accumulation of Connective Tissue Growth Factor Defines a Subset of Reactive Astrocytes, Invading Fibroblasts, and Endothelial Cells Following Central Nervous System Injury in Rats and Humans,” J. NEUROTRAUMA 18(4):377-388 (2001).
Shepard et al. “Delayed secondary glucocorticoid responsiveness of MYOC in human trabecular meshwork cells,” INVEST OPHTHALMOL VIS SCI 42:3173 (2001).
Strong, N. P., “How optometrists screen for glaucoma: a survey,” OPHTHAL PHYSIOL OPT 12:3-7 (1992).
Turner, B. J., et al., “Effect of p75 neurotrophin receptor antagonist on disease progression in transgenic amyotrophic lateral sclerosis mice,” J NEUROSCI RES, 78:193-199 (2004).
van Setten, G. B., T. D. Blalock, et al., “Detection of connective tissue growth factor (CTGF) in human tear fluid: preliminary results,” ACTA OPHTHALMOL SCAND 81:51-53 (2003).
Wang et al., “Optimal procedure for extracting RNA from human ocular tissues and expression profiling of the congenital glaucoma gene FOXC1 using quantitative RT-PCR,” MOL VIS 7:89-94 (2001).

Websites
http://docs.appliedbiosystems.com/pebiodocs/04303859.pdf

We claim:

1. A method for treating CTGF-mediated ocular disorders, said method comprising
a. obtaining an ocular or nonocular tissue or fluid sample from a patient;
b. determining whether said sample contains abnormally high expression or alteration of CTGF gene or its gene product when compared with the amount or sequence of CTGF gene or its gene product in normal samples;
c. administering to a patient exhibiting an abnormally high expression of CTGF a therapeutically effective amount of a composition comprising at least one non-nucleotide or non-protein agent that inhibits CTGF-mediated TrkA/p75^{NTR} receptor signaling, and a pharmaceutically acceptable carrier.

2. The method of claim 1, wherein said sample is an ocular tissue or fluid sample.

3. The method of claim 2, wherein said sample is tear fluid or genomic DNA.

4. The method of claim 1, wherein said administering is by topical application, intracamerally or via an implant.

5. The method of claim 1, wherein the total concentration of said TrkA/p75^{NTR} inhibitor in said composition is from 0.01% to 2%.

6. The method of claim 1, wherein said patient suffers from glaucoma or ocular hypertension.

7. The method of claim 6, wherein said glaucoma is normal-tension glaucoma.

8. The method of claim 1, wherein said agent is K252a.

9. A method for lowering intraocular pressure in a patient in need thereof, said method comprising administering a therapeutically effective amount of a composition comprising at least one agent that inhibits connective tissue growth factor (CTGF)-mediated TrkA/p75^{NTR} receptor signaling, and a pharmaceutically acceptable carrier.

10. The method of claim 9, wherein said administering is by topical application, intracamerally or via an implant.

11. The method of claim 9, wherein the total concentration of said TrkA/p75^{NTR} inhibitor in said composition is from 0.01% to 2%.

12. The method of claim 9, wherein said patient suffers from glaucoma or ocular hypertension.

13. The method of claim 12, wherein said glaucoma is normal-tension glaucoma.

14. The method of claim 9, wherein the agent is K252a.

15. A method for preventing the visual field loss associated with Primary Open Angle Glaucoma (POAG), said method comprising administering to a patient in need thereof a composition comprising at least one agent that inhibits Connective Tissue Growth Factor (CTGF)-mediated TrkA/p75^{NTR} receptor signaling such that intraocular pressure is controlled and protection is provided to retinal ganglion cells or to the optic nerve head.

16. A composition for lowering intraocular pressure and providing neuroprotection in a patient in need thereof, said composition comprising at least one agent that inhibits connective tissue growth factor (CTGF)-mediated TrkA/p75^{NTR} receptor signaling and a pharmaceutically acceptable carrier.

17. The composition of claim 16, wherein the total concentration of said TrkA/p75^{NTR} inhibitor in said composition is from 0.01% to 2%.

18. The composition of claim 16, wherein the TrkA/p75^{NTR} inhibitor is K252a.
